(19) United States
(12) Patent Application Publication
(10) Pub. No.: US 2009/0227554 A1
(43) Pub. Date: Sep. 10, 2009

(54) STABLE LIQUID FORMULATIONS OF ANTI-INFECTIVE AGENTS AND ADJUSTED ANTI-INFECTIVE AGENT DOSING REGIMENS

(75) Inventor: Gary Liversidge, West Chester, PA (US)

Correspondence Address: Fox Rothschild, LLP;Elan Pharma International Limited, 2000 Market Street, Philadelphia, PA, 19103, US

(73) Assignee: ELAN PHARMACEUTICALS, INC., South San Francisco, CA (US)

(21) Appl. No.: 12/397,924

(22) Filed: Mar. 4, 2009

Related U.S. Application Data
(60) Provisional application No. 61/033,598, filed on Mar. 4, 2008.

(51) Int. Cl. A61K 31/546; C12Q 1/02; C12Q 1/18

(52) U.S. Cl. 514/202; 435/29; 435/32

(57) ABSTRACT

Provided are methods of determining a resistance-adjusted dosage regimen of an anti-infective agent for treatment of an infection of a mammal by a resistant infective organism, wherein an effective dosage regimen of the anti-infective agent is known for treatment of an infection of the mammal by a susceptible strain of the infective organism. Methods of treating a cefepime resistant bacterial infection in a patient are also provided.

### RELATIONSHIP TO PRIOR APPLICATIONS

This application claims priority under 35 U.S.C. § 119(e) to U.S. Provisional application 61/033,598 filed on Mar. 4, 2008, and incorporated herein by reference.

### FIELD OF INVENTION

Provided are methods of determining a resistance-adjusted dosage regimen of an anti-infective agent for treatment of an infection of a mammal by a resistant infective organism. Also provided are liquid formulations of anti-infective agents having improved stability.

### BACKGROUND

Resistance to an anti-infective agent is the ability of an infective organism to resist the effects of the anti-infective agent. An example is development of antibiotic resistance in bacteria, the ability of the resistant bacteria to resist the effects of an antibiotic. Antibiotic resistance occurs when bacteria change in some way that reduces or eliminates the effectiveness of anti-bacterial agents, such as antibiotic drugs to cure or prevent infections.

Bacteria can do this through several mechanisms. Some bacteria develop the ability to neutralize the antibiotic before it can do harm, others can rapidly pump the antibiotic out, and still others can change the antibiotic attack site so it cannot affect the function of the bacteria, for example.

Antibiotics kill or inhibit the growth of susceptible bacteria. Sometimes one of the bacteria survives because it has the ability to neutralize or evade the effect of the antibiotic; that one bacterium can then multiply and replace all the bacteria that were killed off by the antibiotic, giving rise to an antibiotic-resistant strain of the bacterial species. Exposure to antibiotics therefore provides selective pressure, which makes the surviving bacteria more likely to be resistant the antibiotic. In addition, bacteria that were at one time susceptible to an antibiotic can acquire resistance through mutation of their genetic material or by acquiring pieces of DNA that code for the resistance properties from other bacteria.

Drug resistance is an especially difficult problem for hospitals harboring critically ill patients who are less able to fight off infections without the help of antibiotics. Use of antibiotics in these patients selects for changes in bacteria that bring about drug resistance. Unfortunately, this worsens the problem by producing bacteria with greater ability to survive even in the presence of strong antibiotics. These even stronger drug-resistant bacteria continue to prey on vulnerable hospital patients.

According to Centers for Disease Control and Prevention (CDC) statistics, nearly 2 million patients in the United States get an infection in the hospital each year; about 90,000 of those patients die each year as a result of their infection, up from 13,300 patient deaths in 1992; more than 70 percent of the bacteria that cause hospital-acquired infections are resistant to at least one of the antibiotics most commonly used to treat them; and people infected with antibiotic-resistant organisms are more likely to have longer hospital stays and require treatment with second- or third-choice medicines that may be less effective, more toxic, and more expensive.

Antimicrobial resistance is driving up health care costs, increasing the severity of disease, and increasing the rates of complications or even death from certain infections, previously effectively treated with antibiotics.

Presently, it is common practice when a patient infected with an anti-infectious agent resistant infectious organism is encountered to not treat that patient's infection with the anti-infectious agent that the infectious organism has developed resistance to. This requires recourse to alternative therapies, such as alternative anti-infectious agents. As more infectious organisms develop resistance to various available anti-infectious agents this situation limits available therapies.

In the hospital setting, intravenous antibiotic therapy is also required for dosing in acutely ill patients who are unable to take oral medicines. In the hospital setting most low bioavailability antibiotics are administered to patients by bolus injection or, more commonly, short intravenous (IV) infusions. Outside the hospital setting portable infusion pumps offer an improvement over bolus antibiotic dosing for some patients, such as cystic fibrosis patients, who require administration of an antibiotic over extended period of days or weeks. Continuous infusion pumps allow a patient to have mobility and to function outside the hospital setting by replacing immobile IV infusion set-ups or repeated bolus dosing in this setting. One problem with extended dosing periods is that the antibiotic may decompose over time or be exposed to temperatures over that which is approved for assuring stability of the antibiotic in solution.

To achieve efficacy in dosing of a cephalosporin against susceptible bacterial strains, a certain target plasma or blood level concentration must be reached to clear the infection caused by a particular bacterial strain. Each strain has an experimentally determined minimum inhibitory concentration (MIC) or minimum bactericidal concentration (MBC) above which an antibiotic has the ability to suppress reproduction (bacteriostatic activity), or kill (bactericidal activity) the organism respectively. Bacteriostatic antibiotics, of which the cephalosporins are a class, at their regularly administered dosages, function by arresting or retarding bacterial growth. MICₛ are usually measured at the fifty percent (50%) level and are experimentally determined by standardized in vitro laboratory tests evaluating activity of antibiotic against a measured inoculum of a bacterial strain susceptible to the antibiotic drug of interest. MIC values are themselves variable and must be experimentally determined for a particular strain of bacteria. A MIC₅₀ is a value determined as the concentration at which a specific organism is reduced by fifty percent. MIC₉₀ indicates that concentration at which there is a ninety percent reduction. “MIC” without further descriptors is usually taken to represent an MIC₅₀ for a specific strain of microorganism. For antibiotic resistant microorganisms, usually a multiple of the non-resistant MIC is necessary for a therapeutic effect against that organism. For example, an antibiotic-resistant bacterium may be determined to have a MIC₅₀ of four times the amount required to treat a non-resistant organism, and multi-drug resistant (MDR) strains may require even higher multiples of the non-resistant MIC.

Beta-lactams are time-dependent antibiotics, meaning that their activity is primarily related to the time during which their serum concentration remains above the MIC for the infecting organism. Thus it has been proposed and used in practice that, in general, longer infusion times have the advantage of maintaining the plasma or blood level of an antibiotic above the MIC for an extended period of time to a short IV infusion. (Craig, et al., Antimicrob. Agents and Chemother. 36 (12): 2577-2583 (1992). Continuous infusions, i.e. infusions that span from one dosage amount to approximately the time for administration of the next dosage, are therefore useful in maintaining blood levels at or above the efficacious concentrations (MIC) for antibiotics with short elimination half-lives such as those that are renally excreted as is the case with MAXIPIME®.

Dosage adjustment increases (i.e. increasing the quantity administered) in short duration or bolus doses, increases the pharmacokinetic absorption curve, thus also increasing the time above MIC, which can enhance the efficacy of bacteriostatic antibiotics. However when more antibiotic is required to be dosed to achieve a similar blood level, there is an increase in the maximal plasma level (or Cₘₐₓ) of the drug, which increases both the risk of toxicity associated with the high maximal blood level, as well as the cost. In contrast, administration regimens that lengthen the dosing period for the antibiotic may actually require lesser amounts of antibiotic to be administered over the same time period. (Craig, et al., Antimicrob. Agents and Chemother. 36 (12): 2577-2583 (1992).

Methods of achieving a sustained plasma level without a higher concentration spike (Cₘₐₓ) include extended or continuous infusions for antibiotics administered parenterally and controlled-release dosage formulations for orally administered antibiotics. As currently taught by the art, most injectable bacteriostatic antibiotics are administered by a short intravenous (IV) infusion with administration times of typically around one-half hour, although the number of references that have studied and/or recommended continuous or extended infusion is growing. MacGowan et al., Clin. Pharmacokinet. 35:391-402 (1998); Tessier et al., Chemotherapy 45:284-295 (1999); Vinks et al., Ther. Drug Monit. 16:341-348 (1994).

The problem that may be associated with extended infusions is the extended period the drug is in solution and the ambient temperature to which the drug is exposed during the administration time. Most parenteral antibiotics are approved for storage and use only at a specified temperature range for a set period of time, usually at or around standard room temperature (between about 20 to about 25 degrees C.). Storage or use at temperature above the approved times and temperature ranges may result in decomposition of the antibiotic into inactive degradants thus lowering the actual dose of active drug thus resulting in safety and efficacy concerns.

For these reasons and others, compositions and methods of treating infections of mammals, including humans, infected with infective organisms are useful.

### SUMMARY OF INVENTION

The methods described herein allow determination of a resistance-adjusted dosage regimen of an anti-infective agent for treatment of an infection of a-mammal by a resistant infective organism.

Provided is a method of determining a resistance-adjusted dosage regimen of an anti-infective agent for treatment of an infection of a mammal by a resistant infective organism. In some embodiments, an effective dosage regimen of the anti-infective agent is known for treatment of an infection of the mammal by a susceptible strain of the infective organism and the method comprises determining the minimum inhibitory concentration (MIC) or minimum lethal concentration (MLC) of the anti-infective agent for the resistant infective organism (MIC_{R} or MLC_{R}); comparing the MIC_{R} or MLC_{R} of the anti-infective agent to the MIC or MLC of the anti-infective agent for the susceptible strain of the infective organism (MICₛ or MLCₛ), to obtain a MIC_{R} to MICₛ ratio or a MLC_{R} to MLCₛ ratio; and adjusting the known dosage regimen to provide the resistance-adjusted dosage regimen. The known dosage regimen is adjusted by modifying a parameter proportionally to the MIC_{R} to MICₛ ratio or MLC_{R} to MLCₛ ratio. That modification allows the anti-infective agent to be effective for treatment of an infection of a mammal by the resistant infective organism.

Also provided is a method of treating an infection of a patient by a resistant infective organism. In some embodiments, that method includes identifying a resistant infective organism infection in a patient; determining a resistance-adjusted dosage regimen of the anti-infective agent for treatment of the infection of the patient by the resistant infective organism according to the method just described; and administering the anti-infective agent to the patient according to the resistance, adjusted dosage regimen to thereby treat the infection of the mammal.

Also provided is a method of treating a cefepime resistant bacterial infection in a patient. In some embodiments the method includes identifying a cefepime resistant bacterial infection in the patient; determining the MIC of cefepime for the resistant bacterial strain (MIC_{R}); determining the ratio of the MICa to the MIC of cefepime for a susceptible strain (MICₛ) of the same bacterial species. (MIC_{R}/MICₛ ratio); determining a modified cefepime dosage regimen using the MIC_{R}/MICₛ ratio, wherein the modified cefepime dosage regimen provides a plasma concentration of cefepime in the patient of at least the MIC_{R} over a period at least about as long as the plasma concentration of cefepime in the patient is at least the MICₛ following administration of cefepime to a patient using an established cefepime dosing regimen; and administering cefepime to the patient according to the modified cefepime dosage regimen, to thereby treat the cefepime resistant bacterial infection in the patient.

Also provided is a method of providing empiric treatment to a febrile neutropenic patient. The method includes identifying a febrile neutropenic patient; initiating treatment of the patient with cefepime using an established cefepime dosing regimen; identifying a cefepime resistant bacterial infection in the patient; determining the MIC of cefepime for the resistant bacterial strain (MIC_{R}); determining the ratio of the MICa to the MIC of cefepime for a susceptible strain (MICₛ) of the same bacterial species (MIC_{R}/MICₛ ratio); determining a modified cefepime dosage regimen using the MIC_{R}/MICₛ ratio, wherein the modified cefepime dosage regimen provides a plasma concentration of cefepime in the patient of at least the MIC_{R} over a period at least about as long as the plasma concentration of cefepime in the patient is at least the MICₛ following administration of cefepime to a patient using the established cefepime dosing regimen; and administering cefepime to the patient according to the modified cefepime dosage regimen, to thereby treat the cefepime resistant bacterial infection in the patient.

In another aspect, the invention provides a stable liquid formulation comprising a cephalosporin antibiotic and a stabilizer. In preferred embodiment, the cephalosporin antibiotic is cefepime and the stabilizer is an acetate buffer. Preferably, the formulation also comprises arginine. The resulting liquid composition preferably has pH of between about 2.5 and about 6.5, more preferably, between about 4.6 and about 5.6.

Also provided is a kit comprising a container having a first compartment comprising a cephalosporin antibiotic and a second compartment comprising an acetate buffer. In an embodiment, the cephalosporin antibiotic is cefepime, and the first compartment further comprises arginine. In an embodiment, the first compartment and the second compartment are configured to be opened into one another. In another embodiment, the first compartment and the second compartment are separate containers.

A method of treatment a disease treatable by cefepime is also provided, the method comprising administering to a patient in need thereof the stable liquid formulation as described above, by intravenous infusion, wherein the duration of the infusion is between about 2 and about 8 hours.

### BRIEF DESCRIPTION OF THE DRAWINGS

FIG. 1 shows a graph of cefepime concentration in the plasma over time for continuous infusion and for a 0.5 hr infusion of a 2 g dose of Maxipime®, and illustrates the period of time that each mode of administration maintains the plasma concentration of a 70 kg subject above the MIC for intermediately resistant and resistant microbes.

### DETAILED DESCRIPTION

For a better understanding of the instant invention, the following non-limiting definitions are provided:

As used herein an “infective organism” is a bacteria, mycobacteria, fungus, protist, or other parasite that infects a mammal.

An “anti-infective agent” is a chemical or biological entity that has the ability to kill an infective organism or to arrest or retard the growth and/or reproduction of the infective organism.

An anti-infective agent is administered by a “dosage regimen.” A dosage regimen includes both a dosage amount and a dosing interval. The dosing interval is the period of time between administration of a first dose and administration of the next dose. In the case of an. anti-infective agent that is administered by infusion, the dosing interval is the time between initiation of administration of a first dose and initiation of administration of the next dose. For example, if an agent is administered by infusion over one hour, with a twelve hour dosing interval, infusion of a first .dose is begun at time zero and completed at about time one hour. Infusion of the next dose is then begun at about time 12 hours and completed at about time 13 hours, etc. In the case of administration by continuous infusion the dosing interval is zero.

The “minimum inhibitory concentration” (MIC) of an anti-infective agent is the concentration above which the agent has the ability to arrest or retard the growth and/or reproduction of an infective organism.

The “minimum lethal concentration” (MLC) of an anti-infective agent is the concentration above which the agent has the ability to kill the infective organism.

The MIC or MLC of an anti-infective agent can differ between one infective organism and another. The MIC or MLC of an anti-infective agent is determined experimentally, by standardized in vitro laboratory tests (“susceptibility tests”), evaluating activity of the anti-infective agent against a measured inoculum of an infective organism strain. The MIC₅₀ is the concentration of anti-infective agent that reduces growth or reproduction of a specific infective organism by fifty percent. “MIC” without further descriptors is used herein to denote an MIC₅₀ for a specific strain of infective organism, unless the context clearly indicates otherwise.

The MLC₅₀ is the concentration of anti-infective agent that kills fifty percent of a specific infective organism. “MLC” without further descriptors is used herein to denote an MLC₅₀ for a specific strain of infective organism, unless the context clearly indicates otherwise.

When an infective organism acquires resistance to an anti-infective agent, the MIC or MLC of the anti-infective agent for that infective organism increases. In this context, the strain of the infective organism prior to acquisition of resistance is defined as “susceptible” Thus, the MIC or MLC of an anti-infective agent for the susceptible strain (MICₛ or MLCₛ) will be lower than the MIC or MLC for the strain that has acquired resistance (MIC_{R} or MLC_{R}). The degree of resistance acquired by a resistant strain can vary. For example, it can vary over time, with the strain becoming resistant to ever higher concentrations of the anti-infective agent over time. Or it can differ between different isolates of the organism. Both forms of variation can and often will exist together in a species of infective organism. As a result, MIC_{R} and MLC_{R} may vary between strains of the same species of infective organism and may also vary over time.

A “time-dependent anti-infective agent” is an anti-infective agent for which efficacy is primarily determined by the amount of time during a dosing interval that the plasma concentration of the agent is above its MIC or MLC.

A “concentration dependent anti-infective agent” is an anti-infective agent for which efficacy is primarily determined by the highest plasma concentration of the agent reached during a dosing interval. Anti-infective agents can be time-dependent, concentration-dependent, or both.

The term “succeptible” refers to infective organisms which are likely to be inhibited if the antimicrobial compound in the blood reaches the concentrations usually achievable using a known dosing regimen of an anti-infective agent, particularly, cefepime hydrochloride.

A report of “Intermediate” indicates that the result should be considered equivocal, and, if the microorganism is not fully susceptible to alternative, clinically feasible drugs, the test should be repeated. This category implies possible clinical applicability in body sites where the drug is physiologically concentrated or in situations where high dosage of drug can be used: This category also provides a buffer zone that prevents small uncontrolled technical factors from causing major discrepancies in interpretation.

A report of “Resistant” indicates that the pathogen is not likely to be inhibited if the antimicrobial compound in the blood reaches the concentrations usually achievable. In the context of the modified dosing regimens and methods of determining modified dosing regimen described, herein, an report of “intermediate” is equivalent to a report of “resistant,” and such a modified dosing regimen can be developed to treat an infection by such a strain.

The term “acetate buffer” refers to an equilibrated aqueous solution of acetic acid and acetate anion adjusted to a desired pH.

The term “Cₘₐₓ” refers to the peak plasma concentration of a compound in a subject or a patient or an averaged value over several subjects.

The term “half-life”, also designated as t ½, refers to the period of time required for the plasma concentration or administered amount of a compound in a subject or patient to be reduced to one-half of a given concentration or amount.

The term “Maxipime®” refers to the commercial preparation of cefepime, a sterile, dry mixture of cefepime (as defined above) and L-arginine.

The term “piggyback” refers to a bottle that is shaped like a large vial. Diluent is added into the vial which contains the desired amount of Maxipime (available in 0.5 g, 1 g and 2 g quantities) and the entire vial (usually around 100 ml in volume) is suspended to infuse the drug rather than reconstituting in an IV bag.

The term “Tₘₐₓ” refers to the time at peak plasma concentration of a compound in a subject or a patient or an averaged value over several subjects.

Provided is a method of determining a resistance-adjusted dosage regimen of an anti-infective agent, for treatment of an infection of a mammal by a resistant infective organism. In embodiments of the method an effective dosage regimen, of the anti-infective agent is known for treatment of an infection of the mammal by a susceptible strain of the infective organism. Some embodiments include determining the minimum inhibitory concentration (MIC) or minimum lethal concentration (MLC) of the anti-infective agent for the resistant infective organism (MIC_{R} or MLC_{R}); comparing the MIC_{R} or MLC_{R} of the anti-infective agent to the MIC or MLC of the anti-infective agent for the susceptible strain of the infective organism (MICₛ or MLCₛ), to obtain a MIC_{R} to MICₛ ratio or a MLC_{R} to MLCₛ ratio; and adjusting the known dosage regimen to provide the resistance-adjusted dosage regimen. The known dosage regimen is adjusted by modifying a parameter proportionally to the MIC_{R} to MICₛ ratio or MLC_{R} to MLCₛ ratio. That modification allows the anti-infective agent to be effective for treatment of an infection of a mammal by the resistant infective organism.

In some embodiments of the method the adjustment is selected from an increase in the dose, a decrease of the dosing interval, and an increase in the dose and decrease in the dosing interval. In some embodiments, the increased dose is the product of the known dose and the MIC_{R} to MICₛ ratio or MLC_{R} to MLCₛ ratio. In some embodiments the length of the decreased dosing interval is the product of the known dosing interval and the inverse of the MIC_{R} to MICₛ ratio or MLC_{R} to MLCₛ ratio.

In some embodiments of the method the resistance-adjusted dosage regimen provides a plasma concentration of the anti-infective agent following administration of the anti-infective agent to the mammal that is above the determined MIC_{R} or MLC_{R} for at least about as long as the plasma concentration of the anti-infective agent is above the known MICₛ or MLCₛ following administration of the anti-infective agent to the mammal according to the known dosage regimen.

In some embodiments of the method the resistance-adjusted dosage regimen provides a plasma concentration time profile exhibiting an area under the curve (AUC) above the determined MIC_{R} or MLC_{R} of the anti-infective agent following administration of the anti-infective agent to the mammal that is at least about as large as the AUC above the known MICₛ or MLCₛ following administration of the anti-infective agent to the mammal according to the known dosage regimen.

In some embodiments of the method the resistance-adjusted dosage regimen provides a peak plasma concentration (Cₘₐₓ) above the determined MIC_{R} or MLC_{R} of the anti-infective agent following administration of the anti-infective agent to the mammal that is at least about as large as the Cₘₐₓ above the known MICₛ or MLCₛ following administration of the anti-infective agent to the mammal according to the known dosage regimen.

In some embodiments of the method the. infective organism is chosen from a bacterium, a mycobacterium, a fungus, and a protist.

In some embodiments of the method the mammal is a human. In some embodiments of the method, the anti-infective agent is an antibiotic.

In some embodiments of the method the antibiotic is a cephalosporin. In some embodiments the cephalosporin antibiotic is chosen from cefixime, cefaclor, cefuroxime axetil, cefpodoxime, cefdinir, cefditoren, cefepime, cefoperazone, cefazolin, cefuroxime sodium and cefotaxime. In some embodiments the infective organism is one or more strain of Enterobacter, Escherichia coli, Klebsiella pneumoniae, Proteus mirabilis, Pseudomonas aeruginosa, Acinetobacter calcoaceticus subsp. Iwoffi, Citrobacter diversus, Citrobacter freundii, Enterobacter agglomerans, Haemophilus influenzae (including beta-lactamase producing strains), Hafnia alvei, Klebsiella oxytoca, Moraxella catarrhalis (including beta-lactamase producing strains), Morganella morganii, Proteus vulgaris, Providencia rettgeri, Providencia stuartii, and Serratia marcescens. In some embodiments the infective organism is one or more strain of Staphylococcus aureus (methicillin-susceptible strains), Streptococcus pneumoniae, Streptococcus pyogenes (Lancefield's Group A streptococci), Viridans group streptococci, Staphylococcus epidermidis (methicillin-susceptible strains only), Staphylococcus saprophyticus, and Streptococcus agalactiae (Lancefield's Group B streptococci).

In some embodiments of the method the infective organism is determined to be resistant by comparing the determined MIC to a known MIC standard that defines resistance.

In some embodiments of the method the infective organism is determined to be resistant by comparing the determined MLC to a known MLC standard that defines resistance.

In some embodiments of the method the MIC or MLC is determined by a diffusion technique.

In some embodiments of the method the MIC or MLC is determined by a dilution technique.

In some embodiments of the method treatment of the mammal with the anti-infective agent using the known dosage regimen is initiated prior to determining the resistance-adjusted dosage regimen.

In some embodiments of the method treatment of the mammal with the anti-infective agent using the known dosage regimen is not initiated prior to determining the resistance-adjusted dosage regimen.

In some embodiments of the method the pharmacokinetics of the anti-infective agent are linear at the dose of anti-infective agent administered in the resistance-adjusted dosage regimen.

In some embodiments of the method the pharmacokinetics of the anti-infective, agent-are not linear at the dose of anti-infective agent administered in the resistance-adjusted dosage regimen.

Also provided is a method of treating an infection of a patient by a resistant infective organism. In some embodiments the method includes identifying a resistant infective organism infection in a patient; determining a resistance-adjusted dosage regimen of the anti-infective agent for treatment of the infection of the patient by the resistant infective organism according to the methods described herein; and administering the anti-infective agent to the patient according to the resistance-adjusted dosage regimen to thereby treat the infection of the mammal.

In some embodiments of the method of treatment, the resistant infective organism infection in the mammal is identified by a method comprising comparing the determined MIC to a known MIC standard that defines resistance.

In some embodiments of the method of treatment, the resistant infective organism infection in the mammal is identified by a method comprising comparing the determined MLC to a known MLC standard that defines resistance.

Also provided is a method of treating a cefepime resistant bacterial infection in a patient. In some embodiments the method includes identifying a cefepime resistant bacterial infection in the patient; determining the MIC of cefepime for the resistant bacterial strain (MIC_{R}); determining the ratio of the MIC_{R} to the MIC of cefepime for a susceptible strain (MICₛ) of the same bacterial species. (MIC_{R}/MICₛ ratio); determining a modified cefepime dosage regimen using the MIC_{R}/MICₛ ratio, wherein the modified cefepime dosage regimen provides a plasma concentration of cefepime in the patient of at least the MIC_{R} over a period at least about as long as the plasma concentration of cefepime in the patient is at least the MICₛ following administration of cefepime to a patient using an established cefepime dosing regimen; and administering cefepime to the patient according to the modified cefepime dosage regimen, to thereby treat the cefepime resistant bacterial infection in the patient.

In some embodiments of the method administration of cefepime according to the modified cefepime dosage regimen provides a plasma concentration of cefepime in the patients plasma of at least the MIC_{R} for from about 70% to about 80% of a dosage interval.

In some embodiments of the method the modified dosage regimen comprises administration of a higher dose of cefepime than that administered by the established cefepime dosage regimen.

In some embodiments of the method the modified dosage regimen comprises administration of cefepime at a shorter dosage interval than the cefepime dosage interval of the established cefepime dosage regimen.

In some embodiments of the method the modified dosage regimen comprises administration of a higher dose of cefepime than that administered by the established cefepime dosage regimen, and administration of cefepime at a shorter dosage interval than the cefepime dosage interval of the established cefepime dosage regimen.

In some embodiments of the method the patient is infected with one or more gram-positive microorganism.

In some embodiments of the method the patient is infected with one or more gram-negative microorganism.

In some embodiments of the method the patient is infected with one or more strain of Enterobacter, Escherichia coli, Klebsiella pneumoniae, Proteus mirabilis, Pseudomonas aeruginosa, Acinetobacter calcoaceticus subsp. Iwoffi, Citrobacter diversus, Citrobacter freundii, Enterobacter agglomerans, Haemophilus influenzae including beta-lactamase producing strains), Hafnia alvei, Klebsiella oxytoca, Moraxella catarrhalis (including beta-lactamase producing strains), Morganella morganii, Proteus vulgaris, Providencia rettgeri, Providencia stuartii, and Serratia marcescens

In some embodiments of the method the patient is infected with one or more strain of Staphylococcus aureus (methicillin-susceptible strains), Streptococcus pneumoniae, Streptococcus pyogenes (Lancefield's Group A streptococci), Viridans group streptococci, Staphylococcus epidermidis (methicillin-susceptible strains only), Staphylococcus saprophyticus, and Streptococcus agalactiae (Lancefield's Group B streptococci).

In some embodiments of the method the patient has moderate to severe pneumonia caused by Streptococcus pneumoniae. In some embodiments the pneumonia is associated with one or more of concurrent bacteremia, infection by Pseudomonas aeruginosa, infection by Klebsiella pneumoniae, and infection by Enterobacter.

In some embodiments of the method the patient is treated for a urinary tract infection. In some embodiments the infection is a severe Escherichia coli or Klebsiella pneumoniae infection. In some embodiments the infection is from a mild to moderate Escherichia coli, Klebsiella pneumoniae, or Proteus mirabilis infection. In some embodiments the infection is associated with concurrent bacteremia.

In some embodiments of the method the infection is an uncomplicated skin or skin structure infection caused by a methicillin-susceptible strain of Staphylococcus aureus or caused by Streptococcus pyogenes.

In some embodiments of the method the infection is a complicated intra-abdominal Escherichia coli, viridans group streptococci, Pseudomonas aeruginosa, Klebsiella pneumoniae, Enterobacter species, or Bacteroides fragilis infection. In some embodiments, the method further comprises administration of metronidazole to the patient.

In some embodiments of the method the MICₛ for the bacterial strain is about 8 ug/mL or less, the MIC_{R} for the bacterial strain is about 32 ug/mL or greater, and the MIC_{R}/MICₛ ratio is at least about 4.

In some embodiments the established cefepime dosage regimen is from 1 to 2 g of cefepime administered intravenously about every 12 hours for a therapeutic dosing period. In some embodiments the therapeutic dosing period if up to about 10 days. In some embodiments the modified cefepime dosage regimen comprises intravenous administration of at least from 4 to 8 g of cefepime every 12 hours for a therapeutic dosing period. In some embodiments the modified cefepime dosage regimen comprises administration of from 1 to 2 g of cefepime intravenously with a dosing interval of 3 hours or less for a therapeutic dosing period.

In some embodiments of the method the established cefepime dosage regimen is 2 g of cefepime administered intravenously about every 12 hours for a therapeutic dosing period. In some embodiments the therapeutic dosing period is up to about 10 days. In some embodiments the modified cefepime dosage regimen comprises administration of at least 8 g of cefepime intravenously every 12 hours for a therapeutic dosing period. In some embodiments the modified cefepime dosage regimen comprises administration of 2 g of cefepime intravenously with a dosing period of three hours or less for a therapeutic dosing period.

In some embodiments of the method the established cefepime dosage regimen is 2 g of cefepime administered intravenously about every 8 hours for a therapeutic dosing period. In some embodiments the therapeutic dosing period is up to about 10 days. In some embodiments the modified cefepime dosage regimen comprises administration of at least 8 g of cefepime intravenously every 8 hours for a therapeutic dosing period. In some embodiments the modified cefepime dosage regimen comprises administration of 2 g of cefepime intravenously with a dosing period of two hours or less for a therapeutic dosing period.

In some embodiments of the method the established cefepime dosage regimen is from 0.5 to 1 g of cefepime administered intravenously or intramuscularly about every 12 hours for a therapeutic dosing period. In some embodiments the therapeutic dosing period if up to about 10 days. In some embodiments the modified cefepime dosage regimen comprises intravenous or intramuscular administration of at least from 2 to 4 g of cefepime every 12 hours for a therapeutic dosing period. In some embodiments the modified cefepime dosage regimen comprises administration of from 0.5 to 1 g of cefepime intravenously or intramuscularly with a dosing interval of 3 hours or less for a therapeutic dosing period.

As used herein “cefepime hydrochloride” refers to the antibiotic approved by the U.S. Food and Drug Administration (FDA) as MAXlPIME® (cefepime hydrochloride, USP) and any cefepime containing composition approved by the FDA on an application citing MAXlPIME® as the listed drug. MAXIPIME® (cefepime hydrochloride) is distributed in the United States by Elan Pharmaceuticals, Inc.

In additional embodiments, more fully described below, cefepime is administered in a prolonged continuous infusion.

MAXIPIME® (cefepime hydrochloride, USP) is a semi-synthetic, broad spectrum, cephalosporin antibiotic for parenteral administration. The chemical name is 1-[[(6R,7R)-7-[2-(2-amino-4-thiazoly-glyoxyamido]-2-carboxy-8-oxo-5-thia-1-azabicyclo[4.2.0]oct-2-en-3-yl]methyl]-1-methylpyrrolidinium chloride, 7²-(Z)(O-methyloxime), monohydrochloride, monohydrate, which corresponds to the following structural formula:

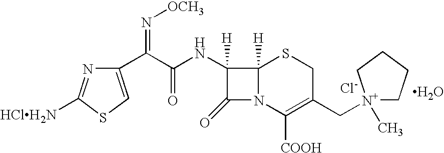

Cefepime hydrochloride MAXIPIME® is a white to pale yellow powder. Cefepime hydrochloride MAXIPIME® contains the equivalent of not less than 825 ug and not more than 911 ug of cefepime (0₁₋H₂₄N₆O₅S₂) per mg, calculated on an anhydrous basis. It is highly soluble in water.

MAXIPIME® is a sterile, dry mixture of Cefepime hydrochloride and L-arginine. It contains the equivalent of not less than 90.0 percent and not more than 115.0 percent of the labeled amount of cefepime (C₁₉H₂₄N₆O₅S₂). The L-arginine, at an approximate concentration of 725 mg/g of cefepime, is added to control the pH of the constituted solution at 4.0-6.0. Freshly constituted solutions of MAXlPIME® will range in color from colorless to amber.

MAXIPIME® (cefepime hydrochloride, USP) for Injection is supplied in 500 mg, 1 g and 2 g doses based on cefepime activity. These dosages are supplied in different containers such as ADD-Vantage® Vials, Piggyback bottles and 15 and 20 mL vials.

An “established cefepime dosing regimen” is a cefepime dosing regimen that has been approved by the FDA and is listed on the MAXIPIME® Prescribing Information.

The current FDA approved adult and pediatric dosage regimens and routes of administration are outlined in Table 1. In those dosage regimens MAXIPIME® is administered intravenously over about 30 minutes.

TABLE 1
Recommended Dosage Schedule for MAXIPIME in Patients with CrCL >60 mL/min

| | | | Duration |
|---|---|---|---|
| Site and Type of Infection | Dose | Frequency | (days) |
| Adults | | | |
| Moderate to Severe Pneumonia due to S. pneumoniae*, | 1-2 g IV | q12 h | 10 |
| P. aeruginosa, K. pneumoniae, or Enterobacter species | | | |
| Empiric therapy forfebrile neutropenic patients (See | 2 g IV | q8 h | 7** |
| INDICATIONS AND USAGE and CLINICAL STUDIES.) | | | |
| Mild to Moderate Uncomplicated or Complicated Urinary Tract | 0.5-1 g | q12 h | 7-10 |
| Infections, including pyelonephritis, due to E. coil, | IV/IM*** | | |
| K. pneumoniae, or P. mirablils* | | | |
| Severe Uncomplicated or Complicated Urinary Tract Infections, | 2 g IV | q12 h | 10 |
| including pyelonephritis, due to E. coil or K. pneumoniae* | | | |
| Moderate to Severe Uncomplicated Skin and Skin Structure | 2 g IV | q12 h | 10 |
| Infections due to S. aureus or S. pyogenes | | | |
| Complicated Intra-abdominal Infections (used in combination | 2 g IV | q12 h | 7-10 |
| with metronidazole (caused by E. coil, viridans group | | | |
| streptococci, P. aeruginosa, K. pneumoniae, Enterobacter species, | | | |
| or B. fragilis. | | | |
| (See CLINICAL STUDIES.) | | | |

Pediatric Patients (2 months up to 16 years)
The maximum dose for pediatric patients should not exceed the recommended adult dose. The usual
recommended dosage in pediatric patients up to 40 kg in weight for uncomplicated and complicated urinary
tract infections (including pyelonephritis), uncomplicated skin and skin structure infections, and pneumonia is
50 mg/kg/dose, administered q12 h (50 mg/kg/dose, q8 h for febrile neutropenic patients) for durations as given
above.
*including cases associated with concurrent bacteremia.
**Or until resolution of neutropenia. In patients whose fever resolves but who remain neutropenic for more than 7 days, the need for continued antimicrobial therapy should be re-evaluated frequently.
***IM route of administration is indicated only for mild to moderate, uncomplicated or complicated UTIs due to E. coli when the IM route is considered to be a more appropriate route of drug administration.

No adjustment is necessary for patients with impaired hepatic function.

In patients with impaired renal function (creatinine clearance ≦60 ml/min), the dose of MAXIPIME® is adjusted to compensate for the slower rate of renal elimination. The recommended initial dose of MAXIPIME® should be the same as in patients-with normal renal function except in patients undergoing hemodialysis. The recommended doses of MAXlPIME® in patients with renal insufficiency are presented in Table 2.

When only serum creatinine is available, the following formula (Cockcroft and Gault equation) may be used to estimate creatinine clearance. The serum creatinine should represent a steady state of renal function:

$$Males \text{:} Creatinine Clearance ( mL \text{/} min ) = \frac{Weight ( kg ) \times ( 140 - age )}{72 \times serum creatinine ( mg \text{/} dL )}$$

Females receive 85% of the males creatinine clearance value.

The current FDA approved adult dosing schedule is varied based on renal function, as shown in Table 2.

TABLE 2
Recommended Dosing Schedule for MAXIPIME ® in Adult Patients
(Normal Renal Function, Renal Insufficiency, and Hemodialysis)

| Creatinine | | | | | | |
|---|---|---|---|---|---|---|
| Clearance (mL/min) | Recommended Maintenance Schedule | | | | | |
| >60 | 500 mg q12 h | 1 g q12 | h | 2 g q12 | h | 2 g q8 h |
| Normal recommended | | | | | | |
| dosing schedule | | | | | | |
| 30-60 | 500 mg q24 h | 1 g q24 | h | 2 g q24 | h | 2 g q12 h |
| 11-29 | 500 mg q24 h | 500 mg q24 | h | 1 g q24 | h | 2 g q24 h |
| <11 | 250 mg q24 h | 250 mg q24 | h | 500 mg q24 | h | 1 g q24 h |
| CAPD | 500 mg q48 h | 1 g q48 | h | 2 g q48 | h | 2 g q48 h |

| | | |
|---|---|---|
| Hemodialysis* | 1 g on day 1, then 500 mg q24 h thereafter | 1 g q24 h |

On hemodialysis days, cefepime should be administered following hemodialysis. Whenever possible, cefepime should be administered at the same time each day.

In patients undergoing continuous ambulatory peritoneal dialysis, MAXIPIME® may be administered at normally recommended doses at a dosage interval of every 48 hours (see Table 2).

In patients undergoing hemodialysis, approximately 68% of the total amount of cefepime present in the body at the start of dialysis will be removed during a 3-hour dialysis period. The dosage of MAXIPIME® for hemodialysis patients is 1 g on Day 1 followed by 500 mg q24 h (every 24 hours) for the treatment of all infections except febrile neutropenia, which is 1 g q24 h. MAXIPIME® should be administered at the same time each day following the completion of hemodialysis on hemodialysis days (see Table 2).

For Intravenous Infusion, the 1 g or 2 g piggyback (100 mL) bottle is constituted with 50 or 100 mL of a compatible IV fluid. Alternatively, the 500 mg, 1 g, or 2 g vial is reconstituted, and an appropriate quantity of the resulting solution is added to an IV container with the compatible IV fluids. The resulting solution is then administered over about 30 minutes.

Additional information regarding administration of MAXIPIME® is available in the prescribing information, which is incorporated herein by reference.

Cefepime is a bactericidal agent that acts by inhibition of bacterial cell wall synthesis. Cefepime has a broad spectrum of in vitro activity that encompasses a wide range of gram-positive and gram-negative bacteria. Cefepime has a low affinity for chromosomally-encoded beta-lactamases. Cefepime is highly resistant to hydrolysis by most beta-lactamases and exhibits rapid penetration into gram-negative bacterial cells. Within bacterial Cells, the molecular targets of cefepime are the penicillin binding proteins (PBP).

Cefepime has been shown to be active against ˜most strains of the following microorganisms, both in vitro and. in clinical infections:

Aerobic Gram-Negative Microorganisms:
Enterobacter
Escherichia coli
Klebsiella pneumoniae
Proteus mirabilis
Pseudomonas aeruginosa

Aerobic Gram-Positive Microorganisms:
Staphylococcus aureus (methicillin-susceptible strains only)
Streptococcus pneumoniae
Streptococcus pyogenes (Lancefield's Group A streptococci)
Viridans group streptococci

Cefepime has been shown to have in vitro activity against most strains of the following microorganisms:

Aerobic Gram-Positive Microorganisms:
Staphylococcus epidermidis (methicillin-susceptible strains only)
Staphylococcus saprophyticus
Streptococcus agalactiae (Lancefield's Group B streptococci)

Aerobic Gram-Negative Microorganisms:
Acinetobacter calcoaceticus subsp. Iwoffi
Citrobacter diversus
Citrobacter freundii
Enterobacter agglomerans
Haemophilus influenzae (including beta-lactamase producing strains)
Hafnia alvei
Klebsiella oxytoca
Moraxella catarrhalis (including beta-lactamase producing strains)
Morganella morganfi
Proteus vulgaris
Providencia rettgeri
Providencia stuartii
Serratia marcescens

Cefepime may be used as described herein to treat an infection with any microorganism that it is active against, whether the microorganism is listed above or not.

Accordingly, provided herein are methods of treating an infection of a mammal by a resistant strain of microorganism and methods of determining a resistance-adjusted dosage regimen, wherein the microorganism is a gram-positive microorganism or a gram-negative microorganism.

In an embodiment, the gram-negative microorganism is, for example and without limitation, one or more strain of Enterobacter, Escherichia coli, Klebsiella pneumoniae, Proteus mirabilis, Pseudomonas aeruginosa, Acinetobacter calcoaceticus subsp. Iwoffi, Citrobacter diversus, Citrobacter freundii, Enterobacter agglomerans, Haemophilus influenzae (including beta-lactamase producing strains), Hafnia alvei, Klebsiella oxytoca, Moraxella catarrhalis (including beta-lactamase producing strains), Morganella morganii, Proteus vulgaris, Providencia rettgeri, Providencia stuartii, and Serratia marcescens.

In an embodiment, the gram-positive microorganism is, for example and without limitation, one or more strain of Staphylococcus aureus (methicillin-susceptible strains), Streptococcus pneumoniae, Streptococcus pyogenes (Lancefield's Group A streptococci), Viridans group streptococci, Staphylococcus epidermidis (methicillin-susceptible strains only), Staphylococcus saprophyticus, and Streptococcus agalactiae (Lancefield's Group B streptococci).

MAXIPIME® is approved for the treatment of the following infections:
Pneumonia (moderate to severe) caused by Streptococcus pneumoniae, including cases associated with concurrent bacteremia; Pseudomonas aeruginosa, Klebsiella pneumoniae, or Enterobacter species;
Uncomplicated and Complicated Urinary Tract Infections (including pyelonephritis) caused by Escherichia coli or Klebsiella pneumoniae, when the infection is severe, or caused by Escherichia coli, Klebsiella pneumoniae, or Proteus mirabilis, when the infection is mild to moderate, including cases associated with concurrent bacteremia with these microorganisms;
Uncomplicated Skin and Skin Structure Infections caused by Staphylococcus aureus (methicillin-susceptible strains only) or Streptococcus pyogenes.
Complicated Intra-abdominal Infections (used in combination with metronidazole) caused by Escherichia coli, viridans group streptococci, Pseudomonas aeruginosa, Klebsiella pneumoniae, Enterobacter species, or Bacteroides fragilis.

MAXlPIME® is also approved for empiric therapy for febrile neutropenic patients.

MICₛ and MLCₛ can be determined using various quantitative techniques, such as dilution techniques and diffusion techniques.

Standardized procedures for the dilution method are, for example, described in National Committee for Clinical Laboratory Standards. Methods for Dilution Antimicrobial Susceptibility Tests for Bacteria that Grow Aerobically—Third Edition. Approved Standard NCCLS Document M7-A3, Vol. 13, No. 25, NCCLS, Villanova, Pa., December 1993). Such methods utilize broth or agar or equivalent with standardized inoculum concentrations and standardized concentrations of the anti-infective agent (e.g., cefepime powder).

In the case of cefepime, in embodiments the MIC values are interpreted according to the following criteria:

TABLE 3

| | MIC (μg/mL) | | |
|---|---|---|---|
| Microorganism | Susceptible (S) | Intermediate (I) | Resistant (R) |
| Microorganisms other | ≦8 | 16 | ≧32 |
| than Haemophilus spp.* | | | |
| and S. pneumonia* | | | |
| Haemophilus spp.* | ≦2 | —* | —* |
| Streptococcus | ≦0.5 | 1 | ≧2 |
| pneumoniae* | | | |

*NOTE:
Isolates from these species should be tested for susceptibility using specialized dilution testing methods. (National Committee for Clinical Laboratory Standards. Methods for Dilution Antimicrobial Susceptibility Tests for Bacteria that Grow Aerobically--Third Edition. Approved Standard NCCLS Document M7-A3, Vol. 13, -No. 25, NCCLS, Villanova, PA, December 1993.) Also, strains of Haemophilus spp. with MICₛ greater than 2 ug/mL should be considered equivocal and should be further evaluated.

Laboratory control infectious organisms may be used as controls when performing a dilution method. Laboratory control infectious organisms are specific strains of infectious organisms with intrinsic biological properties relating to resistance mechanisms and their genetic expression; the specific strains are not clinically significant in their current status.

For example cefepime powder should provide the following MIC values (Table 4) when tested against the designated quality control strains:

TABLE 4

| Microorganism | ATCC | MIC (μg/mL) |
|---|---|---|
| Escherichia coli | 25922 | 0.016-0.12 |
| Staphylococcus aureus | 29213 | 1-4 |
| Pseudomonas aeruginosa | 27853 | 1-4 |
| Haemophilus influenzae | 49247 | 0.5-2 |
| Streptococcus pneumoniae | 49619 | 0.06-0.25 |

Standardized procedures for the diffusion method also provide reproducible, estimates of the susceptibility of infectious organisms, such as bacteria, to anti-infective agents, such as antibiotics. One such standardized procedure requires the use of standardized inoculum concentrations. (National Committee for Clinical Laboratory Standards. Performance Standards for Antimicrobial Disk Susceptibility Tests—Fifth Edition. Approved Standard NCCLS Document M2-A5, Vol. 13, No. 24, NCCLS, Villanova, Pa., December 1993). This procedure uses paper disks impregnated with anti-infectious agent (e.g., 30 ug of cefepime), to test the susceptibility of infectious organisms to the anti-infectious agent (e.g., cefepime). Interpretation is identical to that described above for results using dilution techniques.

For example, reports from such assays providing results of the standard single-disk susceptibility test with a 30-pg cefepime disk are interpreted according to the following criteria:

TABLE 5

| | Zone Diameter (mm) | | |
|---|---|---|---|
| Microorganism | Susceptible (S) | Intermediate (I) | Resistant (R) |
| Microorganisms | ≧18 | 15-17 | ≦14 |
| other than | | | |
| Haemophilus spp.* | | | |
| and S. pneumonia* | | | |
| Haemophilus spp.* | ≧26 | —* | —* |

*NOTE:
Isolates from these species should be tested for susceptibility using specialized diffusion, testing methods. (National Committee for Clinical Laboratory Standards. Performance Standards for Antimicrobial Disk Susceptibility Tests--Fifth Edition. Approved Standard NCCLS Document M2-A5, Vol. 13, No. 24, NCCLS, Villanova, PA, December 1993.) Isolates of Haemophilus spp. with zones smaller than 26 mm should be considered equivocal and should be further evaluated. Isolates of S. pneumoniae should be tested against a 1-pg oxacillin disk; isolates with oxacillin zone sizes larger than or equal to 20 mm may be considered susceptible to cefepime.

As with standardized dilution techniques, diffusion methods require the use of laboratory control infectious organisms to control the technical aspects of the laboratory procedures. Laboratory control infectious organisms are specific strains of infectious organisms with intrinsic biological properties relating to resistance mechanisms and their genetic expression; the specific strains are net clinically significant in their current microbiological status. For the diffusion technique, the 30-pg cefepime disk should provide the following zone diameters in these laboratory test quality control strains (Table 6):

TABLE 6

| Microorganism | ATCC | Zone Size Range (mm) |
|---|---|---|
| Escherichia coli | 25922 | 29-35 |
| Staphylococcus aureus | 25923 | 23-29 |
| Pseudomonas aeruginosa | 27853 | 24-30 |
| Haemophilus influenzae | 49247 | 25-31 |

In another aspect, the invention provides stable compositions comprising a cephalosporin antibiotic, such as, for example, cefepime, as well as beneficial methods of administration of these compositions. Specifically, present invention provides formulations, kits and methods capable of maintaining the stability of cefepime (Maxipime®) at various temperatures for an extended period of time.

In the hospital setting most low bioavailability antibiotics are administered to patients by bolus injection or, more commonly, short intravenous (IV) infusions.

The average plasma concentrations of cefepime observed in healthy adult male volunteers (study subjects) (n=9) at various times following single 30-minute IV infusions of cefepime 500 mg, 1 g, and 2 g are summarized in Table 7.*

TABLE 7 *
Average Plasma Concentrations in μg/mL of
Cefepime and Derived Pharmacokinetic Parameters
(±SD), Intravenous (IV) Administration

| | MAXIPIME | | |
|---|---|---|---|
| Parameter | 500 mg IV | 1 g IV | 2 g IV |
| 0.5 h | 38.2 | 78.7 | 163.1 |
| 1.0 h | 21.6 | 44.5 | 85.8 |
| 2.0 h | 11.6 | 24.3 | 44.8 |
| 4.0 h | 5.0 | 10.5 | 19.2 |
| 8.0 h | 1.4 | 2.4 | 3.9 |
| 12.0 h | 0.2 | 0.6 | 1.1 |
| Cₘₐₓ, μg/mL | 39.1 (3.5) | 81.7 (5.1) | 163.9 (25.3) |
| AUC, h · μg/mL | 70.8 (6.7) | 148.5 (15.1) | 284.8 (30.6) |
| Number of | 9 | 9 | 9 |
| subjects (male) | | | |

* Maxipime product insert

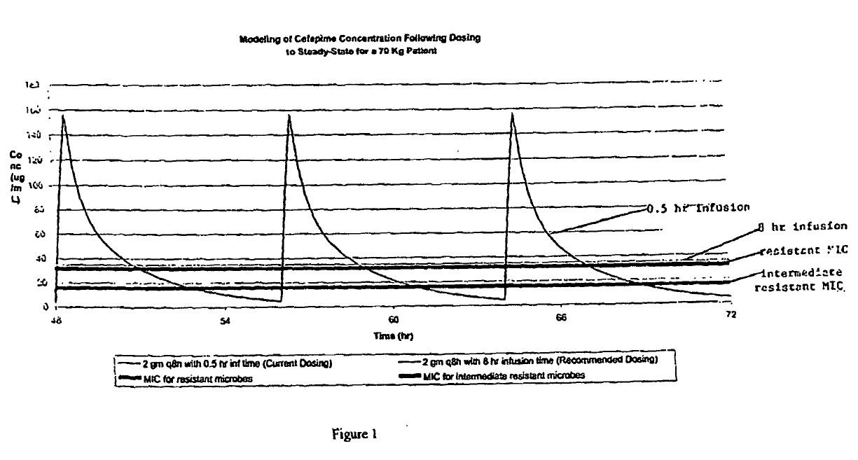

Studies of cefepime's pharmacokinetics, detailed in its product insert, report that elimination of cefepime is principally via renal excretion, which accounts for its rapid elimination, with an average (±SD) half-life of 2.0 (±0.3) hours and total body clearance of 120.0 (±8.0) mL/min in healthy subjects. The rapid clearance is another feature of cefepime pharmacokinetics that makes extended or continuous infusion advantageous. Cefepime pharmacokinetics were linear over the range 250 mg to 2 g. There was no evidence of accumulation in a PK study of healthy adult male volunteers (n=7) receiving clinically relevant doses for a period of 9 days. An approximate graph of the plasma concentration for 0.5 hr infusions is shown in FIG. 1.

Further pharmacokinetic studies using a model of continuous infusion of cefepime has been shown by Monte Carlo simulations to provide drug concentrations above the MIC for resistant, but susceptible microbes at nearly 100 percent of total dosage time once the drug has reached steady state. (FIG. 1)

Even though the mathematical models demonstrate advantages of a continuous prolonged infusion of cefepime may be beneficial, prior art references expressed a concern that the stability of reconstituted MAXIPIME® product during an extended or continuous infusion time may become an issue, see, for example, Scaglione, et al., Expert Rev. Anti. Infect. Ther. 4:479-490 (2006); Soy, et al., Curr. Opin. Crit. Care 12:477-482 (2006). MAXIPIME® reconstituted and used according to current labelling has adequate stability, however there are product reports that document that MAXIPIME® may change color fairly rapidly after reconstitution to give an amber to dark brown solution at ambient room temperature. This discoloration in the reconstituted product in the recommended solution means that the solution may not be used by the clinician for administration. Although not entirely understood what causes the discoloration, it does occur as degradants are formed and detectable in the solution. The addition of acetate buffer may reduce or eliminate decomposition of a reconstituted formulation of MAXIPIME®, which may address this occurrence and also improve stability for usage over extended or continuous infusion times, particularly at temperatures above room temperature.

MAXIPIME®, according to the package insert, is reconstituted from sterile vials, added to about 50 mL to about 100 mL of compatible fluid and then infused over 30 minutes. Suitable compatible fluids are, for example, sterile water for injection, sterile bacteriostatic water for injection with parabens or benzyl alcohol, 0.9% sodium chloride injection, 5% and 10% dextrose injection, M/6 sodium lactate injection, 5% dextrose, lactated Ringers and 5% dextrose injection, Normosol-RTM, and Normosol-MTM in 5% dextrose injection.

The Maxipime package insert provides the following directions for reconstituting and storing the formulation:
“For Intravenous Infusion, constitute the 1 g or 2 g piggyback (100 mL) bottle with 50 or 100 mL of a compatible IV fluid. Alternatively, reconstitution of the 500 mg, 1 g, or 2 g vial, may be done by adding an appropriate quantity of the resulting solution to an IV bag with one of the compatible IV fluids. THE RESULTING SOLUTION SHOULD BE ADMINISTERED OVER APPROXIMATELY 30 MINUTES [emphasis in original]. Intermittent IV infusion with a Y-type administration set can be accomplished with compatible solutions. However, during infusion of a solution containing cefepime, it is desirable to discontinue the other solution. These solutions may be stored up to 24 hours at controlled room temperature 20°-25° C. (680-77° F.) or 7 days in a refrigerator 2°-8° C. (360-46° F.).”

As set forth herein, an improved mode of administration of Maxipime® is by extended or continuous infusion. For the approved dosing interval of 8 hr, an extended or continuous infusion period may extend from about 1 hour to about 8 hr. More preferred is a period of from about 4 hr to about 8 hr and most preferred is a period of about 6 hr to about 8 hr.

Stabilization of the solution may be achieved, for example, in a two (2) gram vial of Maxipime® by addition of about 10 to about 110 mL of about 0.1M to about 0.76 M acetate buffer adjusted to a pH of about 2.5 to about 6.5. In another example there is from about 30 to about 80 mL of acetate buffer in the concentration range of about 0.2M to about 0.5 M with a pH of about 4.6 to about 5.6. In a narrower example, the pH is about 4.6 and molarity of the acetate buffer is about 0.2M.

The pH of the acetate buffer may be adjusted advantageously to more acidic by the addition of a stronger, more concentrated acid than the acetic acid in the solution, which must also be pharmaceutically acceptable, such as hydrochloric acid (HCl). The pH of the acetate buffer may be adjusted advantageously to more basic by the addition of a stronger, more concentrated base than the acetate ion, which must also be pharmaceutically acceptable, such as sodium hydroxide (NaOH). Titration methods for adjustment of the pH of buffer systems are well known to those of skill in the art.

If the above compounding directions are followed for Maxipime as a vial formulation reconstituted with a 0.2 M acetate buffer at pH 4.6 then dilution into a large volume IV container would result in instability attributable to dilution of the buffer and would not be suitable for use for extended or continuous infusion, i.e. greater than 30 minutes. For example, reconstitution of a piggyback formulation with sufficient 0.2 M acetate buffer to provide the desired molarity and pH of about 4.6 to a volume of 50-100 mL followed by infusion according to the current package insert would likely result in vein irritation and acidosis due to infusion of a large volume (50-100 mL or more) of an acidic buffer over a short period of time i.e. 30 minutes. For this reason, extended or continuous infusions and smaller volumes of diluent are preferred. For a further discussion of the influence of pH, buffer catalysis and temperature on cefepime stability see Fubara et al., J. Pharm. Sci. 87:1572-1576 (1998), which is hereby incorporated by reference in its entirety.

In a broad aspect the invention provides a composition for extended or continuous parenteral dosing of a patient in need of antibiotic therapy by continuous infusion of a stabilized Maxipime formulation.

In another aspect of the invention, a composition is provided for safely extending the time period for parenteral dosing of a patient with cefepime/Maxipime at elevated temperatures.

In another aspect, the invention provides a composition for extending the stability of cefepime in a portable continuous infusion pump apparatus.

In yet another aspect a composition comprising an acetate buffer is provided for admixture with a unit dose of cefepime/arginine to provide a formulation having increased stability over time and at temperatures above about 25° C.

Accordingly one embodiment of the invention is a kit comprising a container having a unit dose of about 0.5 to about 2 g of cefepime and another container having an acetate buffer solution that comprises about 10 to about 110 mL of about 0.1M to about 0.76 M acetate buffer adjusted to a pH of about 2.5 to about 6.5.

In another embodiment there is provided a kit comprising a container having a unit dose of about 0.5 to about 2 g of cefepime and another container having an acetate buffer solution that comprises from about 30 to about 80 mL of acetate buffer in a concentration range of about 0.2M to about 0.5 M with a pH of about 4.6 to about 5.6.

In a further embodiment there is provided a kit comprising a container having a unit dose of about 0.5 to about 2 g of cefepime and another container having about 0.2M acetate buffer solution that comprises a solution having a pH of about 4.6, and a volume from about 30 to about 80 mL.

In yet another embodiment, there is provided a kit comprising a unitary sterile container having two or more compartments, one containing a cefepime composition and another containing acetate buffer, wherein the compartments can be opened one to the other to allow mixing of the compartments' contents.

In another aspect of the invention there is provided a formulation comprising a lyophilized composition of cefepime, arginine and acetate buffer in a single container that having an amount of cefepime from about 0.5 g to about 2 g.

Another aspect of the invention provides an article of manufacture comprising: a) a container having a unit dose of about 0.5 to about 2 g of cefepime and another container having an acetate buffer solution; b) printed material providing information on the preparation of the admixture of the cefepime dosage and the acetate buffer; and c) packaging the contains the two containers and printed information.

In another aspect of the invention provides an article of manufacture comprising: a) a container having a unit dose of about 0.5 to about 2 g of cefepime and another container comprising about 10 to about 110 mL of about 0.1M to about 0.76 M acetate buffer adjusted to a pH of about 2.5 to about 6.5; b) printed material providing information on the preparation of the admixture of the cefepime dosage and the acetate buffer; and c) packaging that contains the two containers and the printed information.

In still another aspect the invention provides an article of manufacture comprising: a) a formulation comprising a lyophilized composition of cefepime, arginine and acetate buffer in a single container that having an amount of cefepime from about 0.5 g to about 2 g; b) printed material providing information on the preparation of the admixture of the cefepime dosage and the acetate buffer; and c) packaging that contains the containers and the printed information.

The article of manufacture described herein may contain bulk quantities or less including unit doses of a cefepime/arginine or cefepime/arginine/acetate buffer composition as described herein. The printed material or package insert associated with the container or containers may provide instructions for the use of the composition in treating the condition of choice, instructions for the selecting the dosage amount and for the methods for preparing the composition for administration. The article of manufacture may further comprise multiple containers or compartments, also referred to herein as a kit, comprising a cefepime composition and an acetate buffer, and optionally may further include diluents such as sterile water for injection, sterile bacteriostatic water for injection with parabens or benzyl alcohol, 0.9% sodium chloride injection, phosphate buffered saline (PBS), 5% and 10% dextrose injection, M/6 sodium lactate injection, 5% dextrose, lactated Ringers and 5% dextrose injection, Normosol-RTM, and Normosol-MTM in 5% dextrose injection. It may further include other materials desirable from a commercial and user standpoint, including other buffers, diluents, filters, needles, syringes, and/or package inserts with instructions for use. The cefepime composition can be enclosed in multiple or single dose containers. The cefepime composition and acetate buffer can be provided in kits, optionally including component parts that can be assembled for use. For example, a cefepime composition containing acetate buffer in lyophilized form and a suitable diluent may be provided as separated components for combination prior to use. The article of manufacture may also be a unitary container having separated compartments, one having a cefepime composition and another containing acetate buffer which compartments can access one another and cause mixing of the ingredients.

It will be readily apparent to one of ordinary skill in the relevant arts that other suitable modifications and adaptations to the methods and applications described herein are suitable and may be made without departing from the scope-of the invention or any embodiment thereof. While the invention has been described in connection with certain embodiments, it is not intended to limit the invention to the particular forms set forth, but on the contrary, it is intended to cover such alternatives, modifications and equivalents as may be included within the spirit and scope of the invention as defined by the following claims.

1. A method of determining a resistance-adjusted dosage regimen of an anti-infective agent for treatment of an infection of a mammal by a resistant infective organism, wherein an effective dosage regimen of the anti-infective agent is known for treatment of an infection of the mammal by a susceptible strain of the infective organism, the method comprising:
determining the minimum inhibitory concentration (MIC) or minimum lethal concentration (MLC) of the anti-infective agent for the resistant infective organism (MIC_{R} or MLC_{R});
comparing the MIC_{R} or MLC_{R} of the anti-infective agent to the MIC or MLC of the anti-infective agent for the susceptible strain of the infective organism (MICₛ or MLCₛ), to obtain a MIC_{R} to MICₛ ratio or a MLC_{R} to MLCₛ ratio; and
adjusting the known dosage regimen to provide the resistance-adjusted dosage regimen;
wherein the known dosage regimen is adjusted by modifying a parameter proportionally to the MIC_{R} to MICₛ ratio or MLC_{R} to MLCₛ ratio.

2. The method of claim 1, wherein the adjustment of the known dosage regimen is selected from an increase in the dose, a decrease of the dosing interval, and an increase in the dose and decrease in the dosing interval; or
wherein adjusting the known dosage regimen to provide the resistance-adjusted dosage regimen comprises increasing the dose of the anti-infective agent.

3. The method of claim 2, wherein the increased dose is the product of the known dose and the MIC_{R} to MICₛ ratio or MLC_{R} to MLCₛ ratio.

4. The method of claim 2, wherein the length of the decreased dosing interval is the product of multiplication of the known dosing interval by the inverse of the MIC_{R} to MICₛ ratio or MLC_{R} to MLCₛ ratio.

5. The method of claim 1, wherein the resistance-adjusted dosage regimen provides a plasma concentration of the anti-infective agent following administration of the anti-infective agent to the mammal that is above the determined MIC_{R} or MLC_{R} for at least about as long as the plasma concentration of the anti-infective agent is above the known MICₛ or MLCₛ following administration of the anti-infective agent to the mammal according to the known dosage regimen.

6. The method of claim 1, wherein the resistance-adjusted dosage regimen provides a plasma concentration time profile exhibiting an area under the curve (AUC) above the determined MIC_{R} or MLC_{R} of the anti-infective agent following administration of the anti-infective agent to the mammal that is at least about as large as the AUC above the known MICₛ or MLCₛ following administration of the anti-infective agent to the mammal according to the known dosage regimen.

7. The method of claim 1, wherein the resistance-adjusted dosage regimen provides a peak plasma concentration (Cₘₐₓ) above the determined MIC_{R} or MLC_{R} of the anti-infective agent following administration of the anti-infective agent to the mammal that is at least about as large as the Cₘₐₓ above the known MICₛ or MLCₛ following administration of the anti-infective agent to the mammal according to the known dosage regimen.

8. The method of claim 1, wherein the infective organism is chosen from a bacterium, a mycobacterium, a fungus, and a protist.

9. The method of claim 1, wherein the mammal is a human.

10. The method of claim 1, wherein the anti-infective agent is an antibiotic.

11. The method of claim 10, wherein the antibiotic is a cephalosporin antibiotic.

12. The method of claim 11, wherein the cephalosporin antibiotic is chosen from cefixime, cefaclor, cefuroxime axetil, cefpodoxime, cefdinir, cefditoren, cefepime, cefoperazone, cefazolin, cefuroxime sodium and cefotaxime.

13. The method of claim 11, wherein the infective organism is one or more strain of Enterobacter, Escherichia coli, Klebsiella pneumoniae, Proteus mirabilis, Pseudomonas aeruginosa, Acinetobacter calcoaceticus subsp. Iwoffi, Citrobacter diversus, Citrobacter freundii, Enterobacter agglomerans, Haemophilus influenzae (including beta-lactamase producing strains), Hafnia alvei, Klebsiella oxytoca, Moraxella catarrhalis (including beta-lactamase producing strains), Morganella morganii, Proteus vulgaris, Providencia rettgeri, Providencia stuartii, or Serratia marcescens.

14. The method of claim 11, wherein the. infective organism, is one or more strain of Staphylococcus aureus (methicillin-susceptible strains), Streptococcus pneumoniae, Streptococcus pyogenes (Lancefield's Group A. streptococci), Viridans group streptococci, Staphylococcus epidermidis (methicillin-susceptible strains only), Staphylococcus saprophyticus, or Streptococcus agalactiae (Lancefield's Group B streptococci).

15. The method of claim 1, wherein the infective organism is determined to be resistant by comparing the determined MIC to a known MIC standard that defines resistance.

16. The method of claim 1, wherein the infective organism is determined to be resistant by comparing the determined MLC to a known MLC standard that defines resistance.

17. The method of claim 1 wherein the MIC or MLC is determined by a diffusion technique.

18. The method of claim 1, wherein the MIC or MLC is determined by a dilution technique.

19. The method of claim 1, wherein treatment of the mammal with the anti-infective agent using the known dosage regimen is initiated prior to determining the resistance-adjusted dosage regimen.

20. The-method of claim 1, wherein treatment of the mammal with the anti-infective agent using the known dosage regimen is not initiated prior to determining the resistance-adjusted dosage regimen.

21. The method of claim 1, wherein the pharmacokinetics of the anti-infective agent are linear at the dose of anti-infective agent administered in the resistance-adjusted dosage, regimen.

22. The method of claim 1, wherein the pharmacokinetics of the anti-infective agent are not linear at the dose of anti-infective agent administered in the resistance-adjusted dosage regimen.

23. A method of treating an infection of a patient by a resistant infective organism, comprising:
identifying a resistant infective organism infection in a patient;
determining a resistance-adjusted dosage regimen of the anti-infective agent for treatment of the infection of the patient by the resistant infective organism according to the method of any one of claims 1-22;
and administering the anti-infective agent to the patient according to the resistance-adjusted dosage regimen to thereby treat the infection of the mammal.

24. The method of claim 23, wherein the resistant infective organism infection in the mammal is identified by a method comprising comparing the determined MIC to a known MIC standard that defines resistance.

25. The method of claim 23, wherein the resistant infective organism infection in the mammal is identified by a method comprising comparing the determined MLC to a known MLC standard that defines resistance.

26. A method of treating a cefepime resistant bacterial infection in a patient, comprising:
identifying a cefepime resistant bacterial infection in the patient;
determining the MIC of cefepime for the resistant bacterial strain (MIC_{R});
determining the ratio of the MIC_{R} to the MIC of cefepime for a susceptible strain (MICₛ) of the same bacterial species (MIC_{R}/MICₛ ratio);
determining a modified cefepime dosage regimen using the MIC_{R}/MICₛ ratio, wherein the modified cefepime dosage regimen provides a plasma concentration of cefepime in the patient of at least the MICa over a period at least-about as long as the plasma concentration of cefepime in the patient is at least the MICₛ following administration of cefepime to a patient using an established cefepime dosing regimen;
administering cefepime to the patient according to the modified cefepime dosage regimen, to thereby treat the cefepime resistant bacterial infection in the patient.

27. The method of claim 26, wherein administration of cefepime according to the modified cefepime dosage regimen provides a plasma concentration of cefepime in the patients plasma of at least the MIC_{R} for from about 70% to about 80% of a dosage interval.

28. The method of claim 26, wherein the modified dosage regimen comprises administration of a higher dose of cefepime than that administered by the established cefepime dosage regimen.

29. The method of claim 26, wherein the modified dosage regimen comprises administration of cefepime at a shorter dosage interval than the cefepime dosage interval of the established cefepime dosage regimen.

30. The method of claim 26, wherein the modified dosage regimen comprises administration of a higher dose of cefepime than that administered by the established cefepime dosage regimen, and administration of cefepime at a shorter dosage interval than the cefepime dosage interval of the established cefepime dosage regimen.

31. The method of claim 26, wherein the patient is infected with one or more gram-positive microorganism.

32. The method of claim 26, wherein the patient is infected with one or more gram-negative microorganism.

33. The method of claim 26, wherein the patient is infected with one or more strain of Enterobacter, Escherichia coli, Klebsiella pneumoniae, Proteus mirabilis, Pseudomonas aeruginosa, Acinetobacter calcoaceticus subsp. Iwoffi, Citrobacter diversus, Citrobacter freundii, Enterobacter agglomerans, Haemophilus influenzae (including beta-lactamase producing strains), Hafnia alvei, Klebsiella oxytoca, Moraxella catarrhalis (including beta-lactamase producing strains), Morganella morganii, Proteus vulgaris, Providencia rettgeri, Providencia stuartii, and Serratia marcescens.

34. The method of claim 26, wherein the patient is infected with one or more strain of Staphylococcus aureus (methicillin-susceptible strains), Streptococcus pneumoniae, Streptococcus pyogenes (Lancefield's Group A streptococci), Viridans group streptococci, Staphylococcus epidermidis (methicillin-susceptible strains only), Staphylococcus saprophyticus, and Streptococcus agalactiae (Lancefield's Group B streptococci).

35. The method of claim 26, wherein the patient has moderate to severe pneumonia caused by Streptococcus pneumoniae.

36. The method of claim 35, wherein the pneumonia is associated with one or more of concurrent bacteremia, infection by Pseudomonas aeruginosa, infection by Klebsiella pneumoniae, and infection by Enterobacter.

37. The method of claim 26, wherein the patient is treated for a urinary tract infection.

38. The method of claim 37, wherein the infection is a severe Escherichia coli or Klebsiella pneumoniae infection.

39. The method of claim 37, wherein the infection is from a mild to moderate Escherichia coli, Klebsiella pneumoniae, or Proteus mirabilis infection.

40. The method of claim 39, wherein the infection is associated with concurrent bacteremia.

41. The method of claim 26, wherein the infection is an uncomplicated skin or skin structure infection caused by a methicillin-susceptible strain of Staphylococcus aureus or caused by Streptococcus pyogenes.

42. The method of claim 26, wherein the infection is a complicated intra-abdominal Escherichia coli, viridans group streptococci, Pseudomonas aeruginosa, Klebsiella pneumoniae, Enterobacter species, or Bacteroides fragilis infection.

43. The method of claim 42, further comprising administration of metronidazole to the patient.

44. A method of providing empiric treatment to a febrile neutropenic patient, comprising:
identifying a febrile neutropenic patient;
initiating treatment of the patient with cefepime using an established cefepime dosing regimen;
identifying a cefepime resistant bacterial infection in the patient; determining the MIC of cefepime for the resistant bacterial strain (MIC_{R});
determining the ratio of the MIC_{R} to the MIC of cefepime for a susceptible strain (MICₛ) of the same bacterial species (MIC_{R}/MICₛ ratio);
determining a modified cefepime dosage regimen using the MIC_{R}/MICₛ ratio, wherein the modified cefepime dosage regimen provides a plasma concentration of cefepime in the patient of at least the MIC_{R} over a period at least about as long as the plasma concentration of cefepime in the patient is at least the MICₛ following administration of cefepime to a patient using the established cefepime dosing regimen; and
administering cefepime to the patient according to the modified cefepime dosage regimen, to thereby treat the cefepime resistant bacterial infection in the patient.

45. The method of claim 26, wherein the MICₛ for the bacterial strain is about 8 ug/mL or less, the MIC_{R} for the bacterial strain is about 32 ug/mL or greater, and the MIC_{R}/MICₛ ratio is at least about 4.

46. The method of claim 45, wherein the established cefepime dosage regimen is from 1 to 2 g of cefepime administered intravenously about every 12 hours for a therapeutic dosing period.

47. The method of claim 46, wherein the therapeutic dosing period if up to about 10 days.

48. The method of claim 46, wherein the modified cefepime dosage regimen comprises intravenous administration of at least from 4 to 8 g of cefepime every 12 hours for a therapeutic dosing period.

49. The method of claim 46, wherein the modified cefepime dosage regimen comprises administration of from 1 to 2 g of cefepime intravenously with a dosing interval of 3 hours or less for a therapeutic dosing period.

50. The method of claim 45, wherein the established cefepime dosage regimen is 2 g of cefepime administered intravenously about every 12 hours for a therapeutic dosing period.

51. The method of claim 50, wherein the therapeutic dosing period is up to about 10 days.

52. The method of claim 50, wherein the modified cefepime dosage regimen comprises administration of at least 8 g of cefepime intravenously every 12 hours for a therapeutic dosing period.

53. The method of claim 50, wherein the modified cefepime dosage regimen comprises administration of 2 g of cefepime intravenously with a dosing period of three hours or less for a therapeutic dosing period.

54. The method of claim 45, wherein the established cefepime dosage regimen is 2 g of cefepime administered intravenously about every 8 hours for a therapeutic dosing period.

55. The method of claim 54, wherein the therapeutic dosing period is up to about 10 days.

56. The method of claim 54, wherein the modified cefepime dosage regimen comprises administration of at least 8 g of cefepime intravenously every 8 hours for a therapeutic dosing period.

57. The method of claim 54, wherein the modified cefepime dosage regimen comprises administration of 2 g of cefepime intravenously with a dosing period of two hours or less for a therapeutic dosing period.

58. The method of claim 45, wherein the established cefepime dosage regimen is from 0.5 to 1 g of cefepime administered intravenously or intramuscularly about every 12 hours for a therapeutic dosing period.

59. The method of claim 58, wherein the therapeutic dosing period if up to about 10 days.

60. The method of claim 58, wherein the modified cefepime dosage regimen comprises intravenous or intramuscular administration of at least from 2 to 4 g of cefepime every 12 hours for a therapeutic dosing period.

61. The method of claim 58, wherein the modified cefepime dosage regimen comprises administration of from 0.5 to 1 g of cefepime intravenously or intramuscularly with a dosing interval of 3 hours or less for a therapeutic dosing period.

62. A stable liquid formulation comprising:
a cephalosporin antibiotic or a pharmaceutically acceptable form thereof; and
a stabilizer.

63. The stable liquid formulation of claim 62, wherein the stabilizer comprises an acetate buffer.

64. The stable liquid formulation of claim 62, having pH between about 2.5 and about 6.5.

65. The stable liquid formulation of claim 64, having pH between about 4.6 and about 5.6.

66. The stable liquid formulation of claim 62, wherein the cephalosporin antibiotic is cefepime.

67. The stable liquid formulation of claim 66, comprising between about 0.5 and about 2 g of cefepime.

68. The stable liquid formulation of claim 63, wherein the concentration of the acetate buffer is approximately 0.2M.

69. The stable liquid formulation of claim 62, which does not change its color for about 8 hours after being prepared.

70. The stable liquid formulation of claim 66, further comprising arginine.

71. A kit comprising:
a) a first container comprising a cephalosporin antibiotic or a pharmaceutically acceptable form thereof; and
b) a second container comprising a stabilizer.

72. The kit of claim 71, wherein the cephalosporin antibiotic is cefepime.

73. The kit of claim 71, wherein the first container further comprises arginine.

74. The kit of claim 71, wherein the stabilizer is an acetate buffer.

75. The kit of claim 71 further comprising a set of instructions providing information on the preparation of the admixture of the cefepime dosage and the acetate buffer.

76. A kit comprising a container comprising a first compartment comprising a cephalosporin antibiotic, and a second compartment comprising an acetate buffer, wherein the first compartment and the second compartment are configured to be opened into one another.

77. The kit of claim 76 wherein the first compartment further comprises arginine.

78. A method of treatment of an infection treatable by cefepime, comprising administering to a subject in need thereof by an infusion a stable liquid formulation of claim 62, wherein a duration of the infusion is between about 2 and about 8 hours.
